(19) United States
(12) Patent Application Publication
(10) Pub. No.: US 2006/0122692 A1
(43) Pub. Date: Jun. 8, 2006

(54) Stent valve and method of using same

(76) Inventors: Ran Gilad, Thornhill (CA); Frank Baylis, Beaconsfield (CA); Youssef Biadillah, Montreal (CA)

Correspondence Address: Louis Tessier, C.P. Box 54029, Mount-Royal, QC, H3P 3H4, CA

(21) Appl. No.: 11/250,445

(22) Filed: Oct. 17, 2005

Related U.S. Application Data
(63) Continuation-in-part of application No. 10/841,816, filed on May 10, 2004.
(60) Provisional application No. 60/619,298, filed on Oct. 15, 2004.

(51) Int. Cl. A61F 2/06; A61F 2/90

(52) U.S. Cl. 623/1.24; 623/1.35; 623/1.16

(57) ABSTRACT

A stent valve insertable in a body vessel containing a body fluid. The stent valve includes: a valve for at least partially controlling the flow of the body fluid in the body vessel; and a scaffold, the scaffold including : an anchoring section for anchoring the scaffold to said body vessel and a valve supporting section supporting the valve. The scaffold is substantially radially expandable from a scaffold retracted configuration to a scaffold expanded configuration. The valve supporting section is expandable over a greater range of radial expansion than the anchoring section.

The present invention claims priority from Provisional Application Ser. No. 60/619,298 filed on Oct. 15, 2005. This application is also a Continuation-in-Part of U.S. patent application Ser. No. 10/841,816 filed on May 10, 2004.

I hereby claim the benefit under Title 35, United States Code, § 120, of the prior, co-pending United States application listed herinabove and, insofar as the subject matter of each of the claims of this application is not disclosed in the manner provided by the first paragraph of Title 35, United States Codes § 112, I acknowledge the duty to disclose material information as defined in Title 37, Code of Federal Regulations, § 1.56(a), which occurred between the filing date of this application and the national or PCT international filing date of this application Ser. No. 10/841,816, Filed on May 10, 2004.

### FIELD OF THE INVENTION

The present invention relates to prosthetic devices. More specifically, the present invention is concerned with a stent valve and to a method of using same.

### BACKGROUND OF THE INVENTION

Stent valves are prosthetic devices that typically include a support structure, generally known as a stent, to which a valve is mounted. The stent valve is implantable into a body cavity or body vessel to control the flow of fluid through the cavity or through the vessel. Many stent valves are movable between a retracted and an expanded configuration. In the retracted configuration, the stent valve is insertable percutaneously in a body vessel. Then, the stent valve is positioned to a location at which it is moved to the expanded configuration.

Currently available stent valves are difficult to implement in many locations. For example, stent valves implanted in proximity to a stenotic region of a vessel suffer from relatively poor performance as the valve is then typically expanded to relatively small diameter. Indeed, the valve pressure gradient generated by many types of valve is proportional to the fourth power of <the diameter of the valve at the fourth. Thus, even a relatively a small decreases in diameter would have a tremendous effect on the valve performance.

Another region in which stent valves are relatively hard to position is at bifurcations wherein a vessel bifurcates into two subvessels. Such bifurcations typically have irregular geometries that result in relative difficulties in properly expanding and implanting the stent valve.

While some existing stents that do not include valves may be positioned at similar locations, these stents typically do not take into account constraints caused by the presence of a valve. They are therefore often not readily usable to manufacture stent valves that solve at least some of the above-mentioned problems.

Against this background, there exists a need in the industry to provide a novel stent valve. An object of the present invention is therefore to provide an improved stent valve.

### SUMMARY OF THE INVENTION

In a first broad aspect, the invention provides a stent valve, the stent valve being insertable in a body vessel containing a body fluid. The stent valve includes: a valve for at least partially controlling the flow of the body fluid in the body vessel; and a scaffold, the scaffold including: an anchoring section for anchoring the scaffold to said body vessel and a valve supporting section supporting the valve. The scaffold is substantially radially expandable from a scaffold retracted configuration to a scaffold expanded configuration. The valve supporting section is expandable over a greater range of radial expansion than the anchoring section.

Advantageously, the stent valve is positionable in vessels having irregular geometries. For example, the stent valve is relatively easily positioned substantially adjacent a stenotic region of a blood vessel such that the valve is positioned in a region of the blood vessel that is substantially larger than the stenotic region.

Therefore, the valve and the valve supporting section is relatively easy to extend to a relatively large diameter in the body vessel, which has a potential to improve the performance of the valve as compared to valves that are expandable only to relatively small diameters.

In some embodiments of the invention, the stent valve includes an auxiliary anchoring section allowing anchoring the stent valve at another location in the body vessel. The auxiliary anchoring section helps in reducing the risk that the stent valve will move with respect to the body vessel after it has been moved to the scaffold expanded configuration.

In some embodiments of the invention, the valve supporting section extends directly from the anchoring section. In other embodiments of the invention, the valve supporting and anchoring sections are interconnected by a transition section allowing the valve supporting and anchoring sections to be expanded to different diameters. A suitable transition section typically allows the valve supporting and anchoring sections to be expanded to diameters that differ by a larger amount than the difference allowed in similar stent valves that do not include a transition section. Nevertheless, some embodiments of the invention that do not include the transition section are suitable for many uses.

In some embodiments of the invention, an auxiliary valve anchoring region is usable to anchor against the apex of a bifurcation. Apertures downstream from the valve allow flow to move to the two branches downstream of the bifurcation.

In another broad aspect, the invention provides a stent valve. The stent valve is insertable in a body vessel containing a body fluid. The stent valve includes a valve for at least partially controlling the flow of the body fluid in the body vessel and a scaffold. The scaffold includes an anchoring section for anchoring the scaffold to the body vessel and a valve supporting section supporting the valve. The scaffold is substantially radially expandable between a scaffold retracted configuration and a scaffold expanded configuration, wherein

in the scaffold retracted configuration, the anchoring and valve supporting sections respectively define an anchoring section and a valve supporting section retracted diameter;

in the scaffold expanded configuration, the anchoring and valve supporting sections respectively define an anchoring section and a valve supporting section expanded diameter;

the valve supporting section expanded diameter being greater than said anchoring section expanded diameter.

In yet another broad aspect, the invention provides a method for expanding a stent valve in a body vessel containing a body fluid, the body vessel including a vessel first section having a vessel first section cross-sectional area and a vessel second section having a vessel second section cross-sectional area smaller than the vessel first section cross-sectional area. The stent valve has an anchoring section for anchoring the scaffold to the body vessel and a valve supporting section supporting the valve. The method includes: inserting the stent valve in the body vessel; positioning the stent valve so that the anchoring section is substantially in register with the vessel second section and the valve supporting section is substantially in register with the vessel first section; substantially radially expanding the anchoring section so that the anchoring section anchors the stent valve to the vessel second section; and substantially radially expanding the valve supporting section so that the valve supporting section is substantially radially expanded to extend over a surface having a cross-sectional area larger than the vessel second section cross-sectional area.

Other objects, advantages and features of the present invention will become more apparent upon reading of the following non-restrictive description of preferred embodiments thereof, given by way of example only with reference to the accompanying drawings.

### BRIEF DESCRIPTION OF THE DRAWINGS

In the appended drawings:

FIG. 1, in a side elevation view, illustrates a stent valve in accordance with an embodiment of the present invention, the stent valve being positioned in a body vessel, the stent valve including struts;

FIG. 2, in a side elevation view, illustrates the stent valve of FIG. 1 positioned in an alternative body vessel;

FIG. 3, in a plane view, illustrates an unwrapped view of the stent valve of FIG. 1;

FIG. 4, in a flow chart, illustrates a method for expanding a stent valve in a body vessel;

FIG. 5A, in a side elevation view, illustrates an alternative embodiment of a strut usable in the stent valve of FIG. 1;

FIG. 5B, in a side elevation view, illustrates another alternative embodiment of a strut usable in the stent valve of FIG. 1;

FIG. 5C, in a side elevation view, illustrates yet another alternative embodiment of a strut usable in the stent valve of FIG. 1; and

FIG. 5D, in a side elevation view, illustrates yet another alternative embodiment of a strut usable in the stent valve of FIG. 1.

FIG. 6, in a perspective view, illustrates the stent valve of FIG. 1.

### DETAILED DESCRIPTION

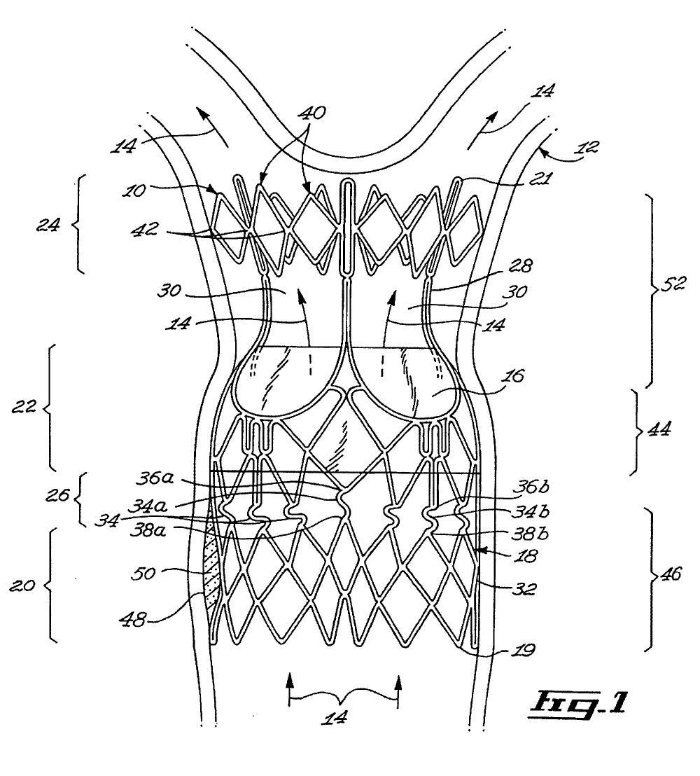

FIG. 1 illustrates a stent valve 10 in accordance with an embodiment of the present invention. The stent valve 10 is insertable in a body vessel 12 containing a body fluid. The movement of the body fluid through the body vessel and through the stent valve 10 is represented by the arrows 14.

The stent valve includes a valve 16 for at least partially controlling the flow of the body fluid in the body vessel 12. The stent valve 10 further includes a scaffold 18. The scaffold 18 is substantially radially expandable from a scaffold retracted configuration (not shown in the drawings) to a scaffold expanded configuration, shown for example in FIG. 1. For example, the scaffold 18 includes interlinked struts 32 that are movable substantially radially so as to allow the scaffold to be moved between the scaffold retracted and expanded configurations.

The scaffold 18 defines a scaffold first longitudinal end 19 and an opposed scaffold second longitudinal end 21. The valve 14 is positioned between the scaffold first and second longitudinal ends 19 and 21.

In some embodiments of the inventions, the valve 14 includes valve leaflets made of a polymeric material, such as for example polyurethane. In these embodiments, a portion of the valve leaflet may embed a portion of the scaffold 18 so as to mount the valve leaflets to the scaffold 18. However, it is within the scope of the invention to include any other suitable valve in the stent valve 10.

The scaffold 18 includes an anchoring section 20 for anchoring the scaffold 18 to the body vessel and a valve supporting section 22 supporting the valve 16. The valve supporting section 22 is expandable over a greater range of radial expansion than the anchoring section 20. Typically, the valve supporting section 20 has a structure that minimizes the risk that the valve 16 could be damaged when the scaffold 18 is moved between the scaffold retracted and expanded configurations.

In some embodiments of the invention, the portion of the valve supporting section 22 to which the valve 16 is mounted expands to a configuration having a substantially smooth configuration. In other words, the curve formed by the struts to which the valve 16 is anchored only includes regions having relatively large radiuses of curvature in the scaffold expanded configuration. This reduces stress concentrations at locations to which the valve 16 is mounted and therefore reduces the risk that the valve 16 is damaged during expansion of the scaffold 18 or during operation of the valve 16 in the body vessel 12. In alternative embodiments of the invention, the portion of the valve supporting section 22 to which the valve 16 is mounted expands to any other suitable configuration.

In some embodiments of the invention, as shown in FIG. 1, the stent valve 10 includes an auxiliary anchoring section 24 for anchoring the scaffold 18 to the body vessel 12. For example, the auxiliary anchoring section 24 is positioned longitudinally opposed to the anchoring section 20 and the valve supporting section is positioned between the anchoring section 20 and the auxiliary anchoring section 24.

In other embodiments of the invention, the scaffold 18 also includes a transition section 26 positioned between the anchoring and valve supporting sections 20 and 22. In yet other embodiments of the invention, the scaffold 18 includes both the auxiliary anchoring section 24 and the transition section 26.

The anchoring and valve supporting sections 20 and 22 are longitudinally spaced apart relative to each other and the transition section 26 extends between the anchoring section 20 and the valve supporting section 22. The transition section 26 is coupled to the valve supporting section 22 and to the anchoring section 20 for allowing the valve supporting section 22 to be expandable over a greater range of radial expansion than the anchoring section 20.

The stent valve 10 shown in FIG. 1 has a valve supporting section 22 that is expandable to a substantially cylindrical configuration. Also, the anchoring section 20 is expandable to a substantially cylindrical configuration. The transition section 26 allows the anchoring and valve supporting sections 20 and 22 to be each expandable to respective substantially cylindrical configurations having different diameters.

In some embodiments of the invention, the anchoring and valve supporting sections 20 and 22 are both simultaneously expandable so as to be both substantially cylindrical. In other embodiments of the invention, the anchoring and valve supporting sections 20 and 22 are sequentially expandable so as to be both substantially cylindrical. In yet other embodiments of the invention, only one of the anchoring and valve supporting sections 20 and 22 is expandable to a substantially cylindrical configuration. In yet other embodiments of the invention, the anchoring and valve supporting sections are expandable to any other suitable configuration.

As shown in FIG. 1, in some embodiments of the invention, the valve supporting section 22 is expandable to a substantially larger diameter than the anchoring section 20. In other words, in the scaffold retracted configuration, the anchoring and valve supporting sections 20 and 22 respectively define an anchoring section and a valve supporting section retracted diameter, and in the scaffold expanded configuration, the anchoring and valve supporting sections 20 and 22 respectively define an anchoring section and a valve supporting section expanded diameter, the valve supporting section expanded diameter being greater than the anchoring section expanded diameter.

In some embodiments of the invention, the diameter of the valve supporting section 22 in the scaffold retracted configuration is substantially smaller than a diameter of the anchoring section 20 in the scaffold retracted configuration. This property may be useful, for example, in embodiments of the invention wherein a valve is stitched to the scaffold 18. The stitching of valve leaflets to scaffolds is well known in the art and will therefore not be described in further details.

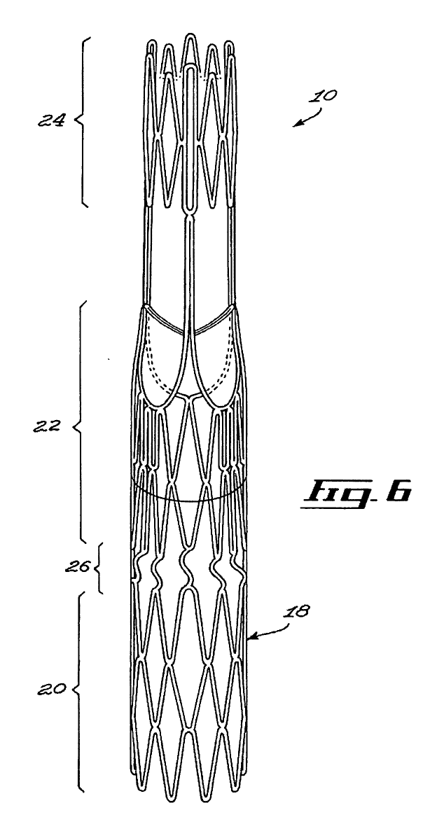

In other embodiments of the invention, as in the stent valve 10, the diameter of the valve supporting section 22 in the scaffold retracted configuration is substantially equal to the diameter of the anchoring section 20 in the scaffold retracted configuration, as shown in FIG. 6.

In addition, as shown in FIG. 1, in some embodiments of the invention, the anchoring and valve supporting sections 20 and 22 are expendable so that they are not necessarily coaxial. However, it is also within the scope of the claimed invention to have the anchoring and valve supporting sections 20 and 22 are expendable so as to be coaxial in the scaffold expanded configuration.

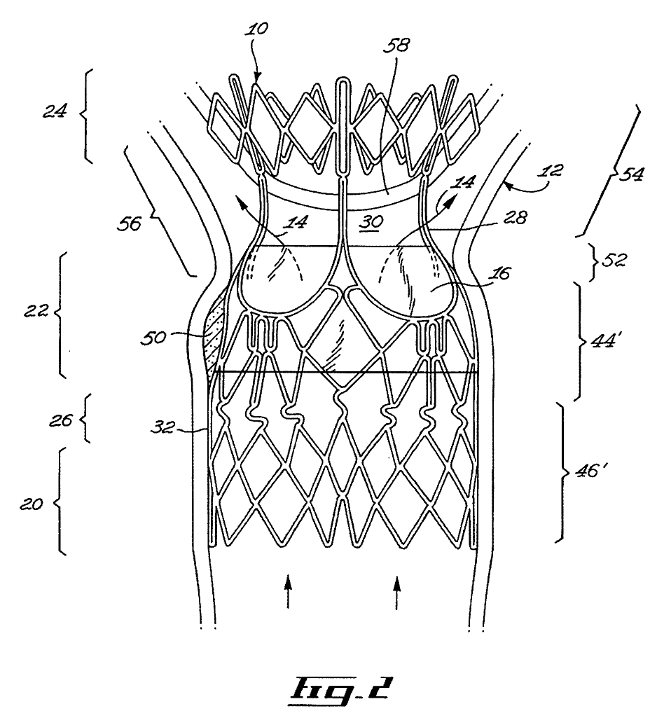

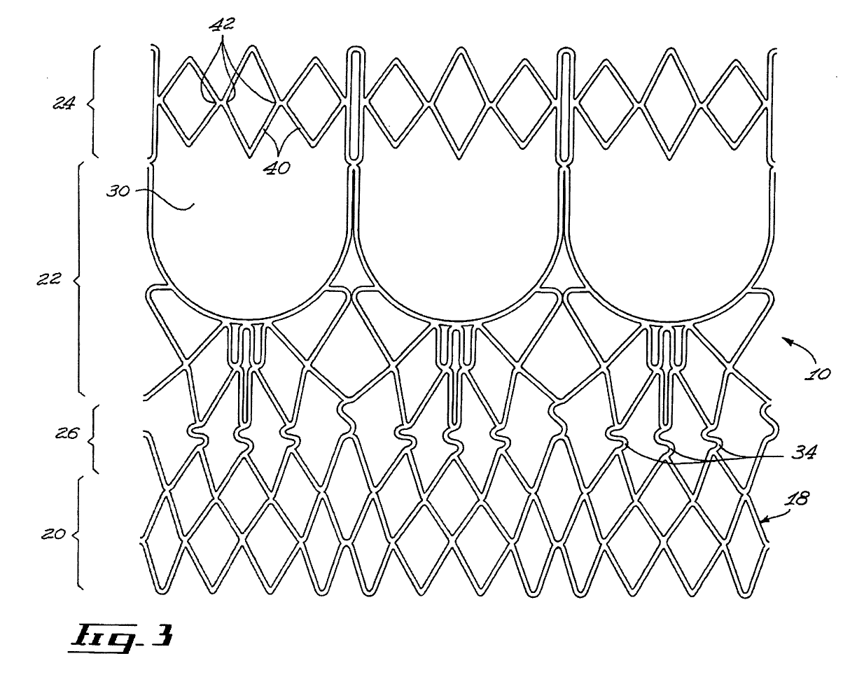

The auxiliary anchoring section 24 is longitudinally spaced apart from the valve supporting section 22. To that effect, the stent valve 10 includes spacing struts 28 extending substantially longitudinally between the auxiliary anchoring section 24 and the valve supporting section 22. In some embodiments of the invention, the spacing struts 28 define at least one radial aperture 30, better illustrated in FIG. 2, for allowing the passage of the body fluid therethrough.

The auxiliary anchoring section 24 is expandable to any suitable configuration, such as for example to a frusto-conical configuration or to a substantially cylindrical configuration, among others. To that effect, the auxiliary anchoring section 24 includes a ring of substantially diamond shaped cells 40, each cell 40 having two circumferentially opposed apexes 42. The diamond shaped cells 40 are interlinked at their circumferentially opposed apexes 42. Therefore, the diamond shaped cells 40 may pivot relative to each other and relative to the valve supporting section 22.

The transition section 26 includes substantially elongated struts collectively designated by the reference numeral 34. The following discussion refers two specific struts, namely struts 34a and 34b. Each of the struts 34a and 34b defines a respective strut first end 36a and 36b and a respective opposed strut second end 38a and 38b.

At least some of the elongated struts 34 are deformable so that their respective strut first and second ends are spaced apart by a greater distance in the scaffold expanded configuration than in the scaffold retracted configuration. For example, the strut 34b shown in FIG. 1 may have a configuration similar to the configuration of the strut 34a when the scaffold 18 is in the scaffold retracted configuration.

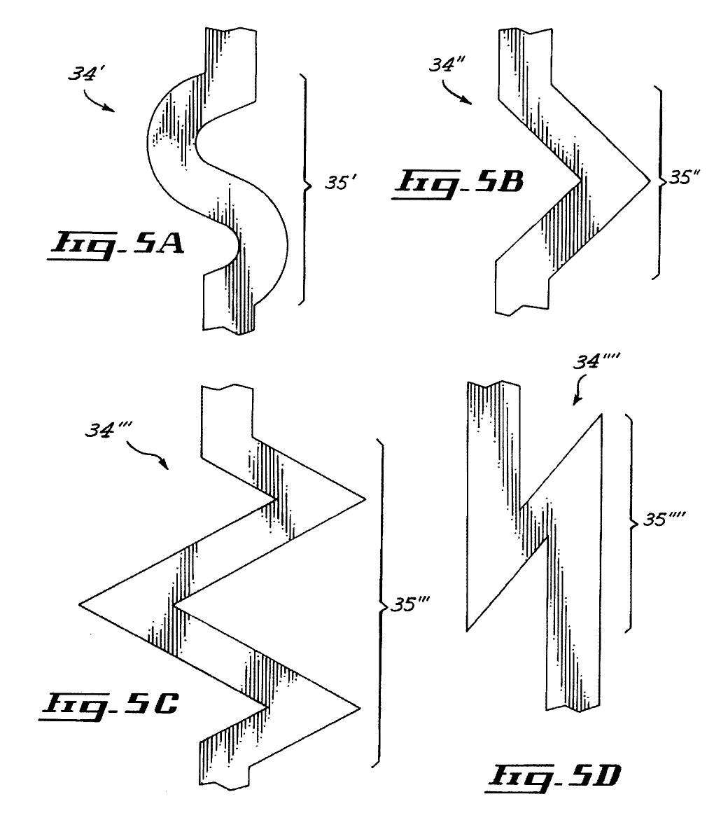

The elongated struts 34 are substantially S-shaped in the scaffold retracted configuration. However, it is within the scope of the invention to have elongated struts 34 having any other suitable configuration. For example, FIGS. 5A, 5B, 5C and 5F respectively illustrate alternative struts 34′, 34″, 34′″ and 34″″ that are usable in alternative transition sections. The struts 34′, 34″, 34′″ and 34″″ include respectively substantially S-shaped, substantially V-shaped, substantially W-shaped, and substantially N-shaped deformable portions 35′, 35″, 35′″ and 35″″. The deformable portions 35′, 35″, 35′″ and 35″″ allow the struts 34′, 34″, 34′″ and 34″″ to be stretched by a relatively large elongation.

The elongated struts 34, 34′, 34″, 34′″ and 34″″ therefore allow the anchoring and valve supporting sections 20 and 22 to be deformed to relatively large differences in diameters while keeping the structural integrity of the stent valve 10. In other words, the deformation of the elongated struts 34, 34′, 34″, 34′″ and 34″″ contributes to the ability of the anchoring and valve supporting sections to have different diameters.

In some embodiments of the invention, the valve supporting and anchoring sections 22 and 20 include different materials. For example, the valve supporting section may include a super elastic material such as for example nitinol. In other embodiments of the invention, another property of nitinol is used and the valve supporting section is self-expandable. However, it is within the scope of the invention to have valve supporting sections 22 that are self expandable but that do not include nitinol. For example, the valve supporting section 22 may be self-expandable because of a specific geometric structure, or it may include an alternative self-expanding material, such as an alternative shape memory material.

The anchoring section 20 may include a balloon expandable material. An example of such a balloon expandable material is stainless steel. It is however within the scope of the invention to use any other suitable material to manufacture the anchoring section.

In some embodiments of the invention, the anchoring section 20 has mechanical properties different from the mechanical properties of the valve supporting section 22. For example, in some embodiments of the invention, the anchoring section 20 is less compressible in a substantially radial direction than the valve supporting section 22.

In embodiments of the invention wherein the anchoring and valve supporting sections 20 and 22 include different materials, the difference in materials may allow the anchoring section 20 to be less compressible in a substantially radial direction than the valve supporting section 22. A similar result may be obtained by changing a thickness in a substantially radial direction of struts forming the anchoring and valve supporting sections 20 and 26.

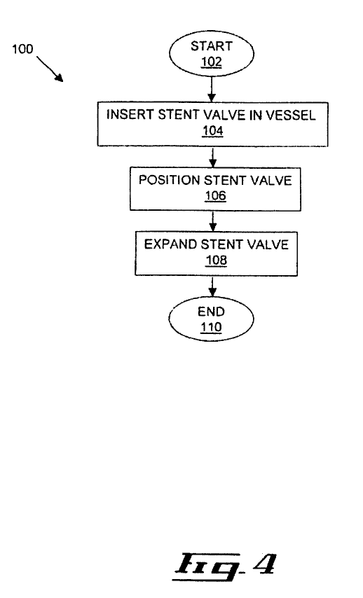

FIG. 4 is a flow chart illustrating a method 100 for expanding the stent valve 10 in the body vessel 12. The body vessel 12 includes a body vessel first section 44 having a vessel first section cross sectional area. The body vessel 12 further includes a vessel second section 46 having a vessel second section cross sectional area smaller than the vessel first section cross sectional area. For clarity, in this document the section cross-sectional areas refer to the cross-sectional area through which the body fluid may flow. For example, if deposits are present on the interior wall of the body vessel, as described hereinbelow, the deposits are taken into account when defining the cross-sectional are of a vessel section in which the deposits are present. Therefore, the cross-sectional area of the vessel is the cross-sectional area of the vessel per se minus the cross-sectional area of occupied by the deposits.

For example, the vessel second section 46 includes a stenotic section 48 wherein the anchoring section 20 is positioned, as described hereinbelow. The stenotic section 48 may be caused at least in part by deposits 50 on the interior surface of the vessel second section 48.

The method starts at step 102. Then, at 104 the stent valve 10 is inserted in the body vessel 12. The stent valve 10 is inserted in the scaffold retracted configuration so as to be able to move relative to the body vessel 12.

Afterwards, at step 106, the stent valve 10 is positioned so that the anchoring section 20 is substantially in register with the vessel second section 46 and the valve supporting section 22 is substantially in register with the vessel first section 44. Subsequently, at step 108, the scaffold 18 is expanded to the scaffold expanded configuration, as illustrated in FIG. 1. In the scaffold expanded configuration, the anchoring section 20 anchors the stent valve 20 to the vessel second section 46. The valve supporting section 22 is substantially radially expanded to expand over a surface having a cross-sectional area larger than the vessel second section cross-sectional area.

In embodiments of the invention wherein the vessel second section includes a stenotic section 48, the anchoring section 20 may be positioned so that it at least partially contacts the stenotic section 48 when expanded. In some embodiments of the invention, the anchoring section crushes the deposits 50 when the scaffold 18 is expanded to the scaffold expanded configuration.

In some embodiments of the invention, anchoring of the anchoring section 20 and auxiliary anchoring section 24 occurs through longitudinal forces exerted by the body vessel 12 substantially parallel to the surface defined of the anchoring and auxiliary anchoring sections 20 and 24.

Finally, the method ends at step 110.

Although not present in all embodiments of the invention, the body vessel 12 includes a vessel third section 52 extending substantially downstream from the vessel first and second sections 46 and 44. The vessel third section 52 is substantially funnel shaped with a minimal diameter smaller than the diameter of the vessel second section 46. The shape of the vessel third section 52 is provided only for example purposes and should not constrain the scope of the claimed invention.

In this embodiment of the invention, and in similar cases wherein suitable vessel shapes are present in locations adjacent to the stenotic section 48, the auxiliary anchoring section 24 is substantially radially expanded so that it is positioned in the vessel third section 52. In some embodiments of the invention, the vessel third section 52 continuous with the vessel first section and may therefore not be distinguished therefrom.

In other embodiments of the invention, as better shown in FIG. 2, the body vessel 12 bifurcates in a vessel fourth section 54 and a vessel fifth section 56. The vessel fourth and fifth sections 54 and 56 extend from the vessel third section 52. The vessel fourth and fifth sections 54 and 56 intersect at a vessel bifurcation apex 58.

The stent valve 10 is positionable so that the auxiliary anchoring section 24 is substantially adjacent to the vessel bifurcation apex 58 prior to being expanded. As it is expanded, the auxiliary anchoring section 24 is deformed as to anchor the stent valve 10 to the vessel bifurcation apex 58. The stent valve 10 is prevented from moving in a substantially longitudinal direction by forces exerted onto the stent valve 10 in a direction that is not substantially parallel to the surface of the scaffold 12. Indeed, in these embodiments of the invention, contact forces normal to the fourth and fifth sections 54 and 56 help in preventing longitudinal movements of the stent valve 10.

As shown in FIG. 2, it may be the case that an alternative vessel second section 44′ is of a diameter smaller than an alternative vessel vessel first section 46′. Accordingly, in these embodiments of the invention, the valve supporting section 22 is not necessarily expanded to a diameter larger than the anchoring section 20.

Although the present invention has been described hereinabove by way of preferred embodiments thereof, it can be modified, without departing from the spirit and nature of the subject invention as defined in the appended claims.

What is claimed is:

1. A stent valve, said stent valve being insertable in a body vessel containing a body fluid, said stent valve comprising:
a valve for at least partially controlling the flow of the body fluid in the body vessel; and
a scaffold, said scaffold including
an anchoring section for anchoring said scaffold to said body vessel; and
a valve supporting section supporting said valve;
said scaffold being substantially radially expandable from a scaffold retracted configuration to a scaffold expanded configuration;
wherein said valve supporting section is expandable over a greater range of radial expansion than said anchoring section.

2. A stent valve as defined in claim 1, wherein said anchoring and valve supporting sections are longitudinally spaced apart relative to each other, said stent valve further comprising a transition section extending between said anchoring section and said valve supporting section, said transition section being operatively coupled to said valve supporting section and to said anchoring section for allowing said valve supporting section to be expandable over a greater range of radial expansion than said anchoring section.

3. A stent valve as defined in claim 2, wherein said transition section includes substantially elongated struts each defining a strut first end and an opposed strut second end, at least some of said elongated struts being deformable so that their respective strut first and second ends are spaced apart by a greater distance in said scaffold expanded configuration than in said scaffold retracted configuration.

4. A stent valve as defined in claim 3, wherein at least some of said elongated struts include a deformable portion selected from a substantially S-shaped portion, a substantially W-shaped portion, a substantially N-shaped portion, or a substantially V-shaped portion.

5. A stent valve as defined in claim 2, wherein said valve supporting section is expandable to a substantially cylindrical configuration.

6. A stent valve as defined in claim 2, wherein said anchoring section is expandable to a substantially cylindrical configuration.

7. A stent valve as defined in claim 2, wherein said valve supporting section and said anchoring section are expandable so as to be both substantially cylindrical.

8. A stent valve as defined in claim 7, wherein said valve supporting section and said anchoring section are simultaneously expandable so as to be both substantially cylindrical.

9. A stent valve as defined in claim 1, wherein said valve supporting section is expandable to a substantially larger diameter than said anchoring section.

10. A stent valve as defined in claim 1, wherein a diameter of said valve supporting section in said retracted configuration is substantially smaller than a diameter of said anchoring section in said retracted configuration.

11. A stent valve as defined in claim 1, wherein a diameter of said valve supporting section in said retracted configuration is substantially equal to a diameter of said anchoring section in said retracted configuration.

12. A stent valve as defined in claim 1, further comprising an auxiliary anchoring section for anchoring said scaffold to said body vessel.

13. A stent valve as defined in claim 12, wherein said auxiliary anchoring section is longitudinally spaced apart from said valve supporting section.

14. A stent valve as defined in claim 13, further comprising spacing struts positioned between said auxiliary anchoring section and said valve supporting section.

15. A stent valve as defined in claim 14, wherein said spacing struts extend substantially longitudinally between said auxiliary anchoring section and said valve supporting section.

16. A stent valve as defined in claim 14, further comprising at least one substantially radial aperture for allowing the passage of the body fluid therethrough, said at least one radial aperture being positioned between said valve supporting section and said auxiliary anchoring section.

17. A stent valve as defined in claim 12, wherein said valve supporting section is positioned between said anchoring section and said auxiliary anchoring section.

18. A stent valve as defined in claim 12, wherein said auxiliary anchoring section is deformable to a substantially cylindrical configuration.

19. A stent valve as defined in claim 12, wherein said auxiliary anchoring section is deformable to a substantially frusto-conical configuration.

20. A stent valve as defined in claim 12, wherein said auxiliary anchoring section includes a ring of substantially diamond-shaped cells each having two circumferentially opposed apexes, said diamond-shaped cells being interlinked at said circumferentially opposed apexes.

21. A stent valve as defined in claim 1, wherein at least one of said anchoring and valve supporting sections includes interlinked struts.

22. A stent valve as defined in claim 1, wherein said valve supporting section includes a superelastic material.

23. A stent valve as defined in claim 1, wherein said valve supporting section is self-expandable.

24. A stent valve as defined in claim 23, wherein said valve supporting section includes a self-expanding material.

25. A stent valve as defined in claim 24, wherein said self-expanding material includes a shape memory material.

26. A stent valve as defined in claim 25, wherein said shape memory material includes a nitinol.

27. A stent valve as defined in claim 1, wherein said anchoring section includes a balloon-expandable material.

28. A stent valve as defined in claim 27, wherein said balloon-expandable material includes stainless steel.

29. A stent valve as defined in claim 27, wherein in said anchoring section is expandable to a non-cylindrical configuration.

30. A stent valve as defined in claim 1, wherein said anchoring section has mechanical properties different from the mechanical properties of said valve supporting section.

31. A stent valve as defined in claim 30, wherein said anchoring section is less compressible in a substantially radial direction than said valve supporting section.

32. A stent valve as defined in claim 30, wherein said anchoring and valve supporting sections include different materials allowing said anchoring section to be less compressible in a substantially radial direction than said valve supporting section.

33. A stent valve as defined in claim 30, wherein said anchoring and valve supporting sections have different thicknesses in a substantially radial direction, said different thicknesses causing at least in part said anchoring section to be less compressible in a substantially radial direction than said valve supporting section.

34. A stent valve as defined in claim 1, wherein said scaffold defines a first scaffold longitudinal end and an opposed second scaffold longitudinal end, said valve being positioned between said first and second scaffold longitudinal ends.

35. A stent valve, said stent valve being insertable in a body vessel containing a body fluid, said stent valve comprising:
a valve for at least partially controlling the flow of the body fluid in the body vessel; and
a scaffold, said scaffold including
an anchoring section for anchoring said scaffold to said body vessel;
a valve supporting section supporting said valve;
said scaffold being substantially radially expandable between a scaffold retracted configuration and a scaffold expanded configuration, wherein
in said scaffold retracted configuration, said anchoring and valve supporting sections respectively define an anchoring section and a valve supporting section retracted diameter;
in said expanded configuration, said anchoring and valve supporting sections respectively define an anchoring section and a valve supporting section expanded diameter;
said valve supporting section expanded diameter is greater than said anchoring section expanded diameter.

36. A method for expanding a stent valve in a body vessel containing a body fluid, the body vessel including a vessel first section having a vessel first section cross-sectional area and a vessel second section having a vessel second section cross-sectional area smaller than the vessel first section cross-sectional area, the stent valve having an anchoring section for anchoring the scaffold to the body vessel and a valve supporting section supporting the valve, said method comprising:
inserting the stent valve in the body vessel;
positioning the stent valve so that the anchoring section is substantially in register with the vessel second section and the valve supporting section is substantially in register with the vessel first section;
substantially radially expanding the anchoring section so that the anchoring section anchors the stent valve to the vessel second section; and
substantially radially expanding the valve supporting section so that the valve supporting section is substantially radially expanded to extend over a surface having a cross-sectional area larger than the vessel second section cross-sectional area.

37. A method as defined in claim 36, wherein the vessel second section includes a stenotic section and the anchoring section is positioned to at least partially contact the stenotic section when the anchoring section is expanded.

38. A method as defined in claim 37, wherein the stenotic section is caused at least in part by deposits on the interior surface of the vessel second section, said method further comprising crushing the deposits when expanding the anchoring section.

39. A method as defined in claim 38, wherein the stent valve includes an auxiliary anchoring section, the auxiliary anchoring section being substantially radially expandable, said method further comprising
positioning the auxiliary anchoring section substantially in register with the vessel first section; and
substantially radially expanding the auxiliary anchoring section so that the auxiliary anchoring section anchors the stent valve to the vessel first section.

40. A method as defined in claim 38, wherein
the stent valve includes an auxiliary anchoring section, the auxiliary anchoring section being substantially radially expandable;
the body vessel includes a third vessel section and a fourth vessel section, the third and fourth vessel sections extending from the second vessel section so as to form a bifurcation of the second vessel section;
the third and fourth vessel sections intersecting at a vessel bifurcation apex;
said method further comprising
positioning the auxiliary anchoring section substantially adjacent the vessel bifurcation apex; and
deforming the auxiliary anchoring section so that the auxiliary anchoring section anchors the stent valve to the vessel bifurcation.
